(19) United States
(12) Patent Application Publication
(10) Pub. No.: US 2010/0240720 A1
(43) Pub. Date: Sep. 23, 2010

(54) ALLOSTERIC JNK INHIBITORS

(75) Inventor: Maurizio Pellecchia, San Diego, CA (US)

Correspondence Address: DLA PIPER LLP (US), 4365 EXECUTIVE DRIVE, SUITE 1100, SAN DIEGO, CA, 92121-2133, US

(73) Assignee: Burnham Institute for Medical Research, La Jolla, CA (US)

(21) Appl. No.: 12/728,093

(22) Filed: Mar. 19, 2010

Related U.S. Application Data
(60) Provisional application No. 61/162,186, filed on Mar. 20, 2009.

(51) Int. Cl. A61K 31/427; C07D 333/38; A61K 31/381; C07D 409/12; C07D 277/30; C07D 403/12; A61K 31/416; A61P 25/00; A61P 25/16; A61P 25/28

(52) U.S. Cl. 514/365; 549/69; 514/447; 549/59; 514/444; 548/204; 549/60; 548/306.1; 514/403; 549/58; 514/443

(57) ABSTRACT

The disclosure provides compounds and compositions, and methods of using these compounds and compositions, for the targeted delivery of chemotherapeutic agents.

### CROSS REFERENCE TO RELATED APPLICATIONS

This application claims the benefit of priority under 35 U.S.C. §119(e) to U.S. Provisional Application No. 61/162,186, filed Mar. 20, 2009, the disclosure of which is hereby incorporated by reference in its entirety for all purposes.

### BACKGROUND OF THE DISCLOSURE

1. Field of the Disclosure

The disclosure generally relates to compounds and compositions, and methods of using these compounds and compositions, for the inhibition of kinases, and more specifically, to allosteric JNK inhibitors.

2. Background Information

c-Jun N-terminal kinases (JNKs) are members of the mitogen-activated protein kinase (MAPK) family. JNKs are involved in response to various stimuli, including proinflammatory cytokines and environmental stress. JNKs, and JNK3 in particular, play an important role during apoptotic death of cells and therefore have been implicated in various disorders including stroke, traumatic brain injury and other neurodegenerative diseases such as Parkinson disease, Alzheimer disease and others. Since JNK activity is a physiological regulator of AP-1 transcriptional activity, JNK inhibitors are also expected to reduce inflammatory response.

Apoptosis is a form of cell death in which the cell actively participates in its own destruction in a process involving a characteristic series of biochemical and morphological changes, which are regulated by specific cell death genes. The apoptotic cell death is a process that has been observed in the developing mammalian nervous system. In mice, the inactivation by homologous recombination of genes that encode proteins that promote apoptosis, such as the caspase-3 or the Bax protein, prevents developmental neuronal cell death. The destruction of genes that encode cell death suppressors such as Bcl-x, leads to enhanced neuronal cell death. There is increasing evidence that apoptosis plays an important role in the pathology of acute and chronic neurodegenerative diseases. For example, in transgenic mice overexpressing the anti-apoptotic Bcl-2 protein in the nervous system there is a decrease in infarct volume following cerebral ischemia. Similarly, injection of the caspase inhibitor BAF reduces neuronal cell death following hypoxia/ischaemia in neonatal rats. Another example is spinal muscular atrophy (a motor neuron disease) where loss of function mutations in the SMN gene is associated with the disease. Recent data has shown that the wild type SMN protein binds to Bcl-2 and co-operates with it to inhibit apoptosis. These results suggest that inhibitors of neuronal apoptosis could be beneficial in the treatment of human neurodegenerative diseases. There is increasing evidence that neuronal apoptosis is an important pathological feature of stroke, traumatic brain injury and other neurodegenerative diseases. Therefore, pharmacotherapy using inhibitors of neuronal apoptosis may provide a therapeutic benefit in neurodegenerative conditions.

A number of groups have studied the mechanisms of neuronal cell death using in vitro cell culture systems and the results suggest that in some systems the transcription factor c-Jun is activated by the removal of survival signals and promotes cell death. c-Jun is activated by JNKs, which phosphorylate its transcriptional activation domain. In humans there are three JNK genes: JNK1, JNK2 and JNK3. The RNAs encoding JNK1 and JNK2 are expressed in many tissues, including the brain, but JNK3 is restricted to the nervous system and to a smaller extent the heart and testes. JNKs are strongly activated in cellular responses to various stresses such as UV radiation, heat shock, osmotic shock, DNA-damaging agents, and proinflammatory cytokines such as TNF-α, IL-1β and others. Upstream regulators of the JNK pathway include kinases such as SEK1, MKK7 and MEKK1. There is evidence that Jun kinase activity is required for neuronal apoptosis in vitro. Overexpression of MEKK1 in sympathetic neurones increased c-Jun protein levels and phosphorylation and induced apoptosis in the presence of NGF indicating that activation of the Jun kinase pathway can trigger neuronal cell death. The Jun kinase pathway has been shown to be necessary for the death of differentiated PC12 cells deprived of NGF. Furthermore, compound CEP-1347, which inhibits the c-Jun pathway (upstream of Jun kinase), protects motor neurones against cell death induced by survival factor withdrawal.

Inhibiting kinases, such as JNK, is one method of treating various diseases, disorders and pathologies. Previously, compounds that are useful as inhibitors of certain kinases have been identified and synthesized that target the ATP binding site of the protein kinase. However, no compounds have been reported that are capable of targeting and inhibiting JNK kinase binding to the docking site (JIP site) for the substrate or scaffolding proteins. Accordingly, bi-dentate compounds are needed that are potent and selective against JNK, for example, due to being capable of binding to the JIP-site and the ATP. Currently, there is a need for new, potent, and selective agents for the treatment of various diseases, disorders and pathologies, such as tumors, as well as for the pharmaceutical compositions including such agents. Such agents can be based on inhibitors of certain kinases, such as JNK kinase. The disclosure addresses these issues and further provides related advantages.

### SUMMARY OF THE DISCLOSURE

The disclosure provides compounds, compositions and methods for treating various diseases and pathologies such as cancer, diabetes, and neurological disorders, with allosteric inhibition of JNKs.

Thus, in one aspect the disclosure provides a compound of Formula I:

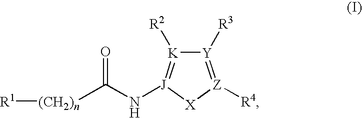

or a pharmaceutically acceptable salt, solvate or hydrate thereof, wherein:

J is independently N or C;

K is independently N or CR²;

Y is independently N or CR³;

Z is independently N or CR⁴;

X is independently O, S, SO, SO₂, or NR⁵;

R¹, R², R³, and R⁴ are each independently hydrogen, halogen, cyano, nitro, hydroxyl, substituted or unsubstituted alkyl, substituted or unsubstituted alkene, substituted or unsubstituted alkyne, substituted or unsubstituted haloalkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, substituted or unsubstituted alkylaryl, substituted or unsubstituted heteroalkylaryl, —(CH₂)ⱼC(═Z)R⁶, —(CH₂)ⱼOR⁶, —(CH₂)ⱼC(O)R⁶, —(CH₂)ⱼC(O)OR⁶, —(CH₂)ⱼNR⁷R⁸, —(CH₂)ⱼC(O)NR⁷R⁸, —(CH₂)ⱼOC(O)NR⁷R⁸, —(CH₂)ⱼNR⁹C(O)R⁶, —(CH₂)ⱼNR⁹C(O)OR⁶, —(CH₂)ⱼNR⁹C(O)NR⁷R⁸, —(CH₂)ⱼS(O)ₖR¹⁰, —(CH₂)ⱼNR⁹S(O)₂R¹⁰, or —(CH₂)ⱼS(O)₂NR⁷R⁸; wherein each j is independently an integer from 0, 1, 2, 3, 4, 5 to 6; and k is independently an integer from 0, 1 to 2; and Z is O, S or NR¹¹;

R⁵ is independently hydrogen or alkyl;

R⁶ is independently hydrogen, substituted or unsubstituted alkyl, substituted or unsubstituted haloalkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁷, R⁸, R⁹ and R¹⁰ are each independently hydrogen, substituted or unsubstituted alkyl, substituted or unsubstituted alkyl-NR¹²R¹³, substituted or unsubstituted alkyl-CONR¹²R¹³, substituted or unsubstituted heteroalkyl, substituted or unsubstituted cycloalkyl, substituted or unsubstituted heterocycloalkyl, substituted or unsubstituted aryl, or substituted or unsubstituted heteroaryl, substituted or unsubstituted aralkyl, substituted or unsubstituted heteroaralkyl, or R⁷ and R⁸, together with the nitrogen to which they are attached, form substituted or unsubstituted 3- to 7-membered heterocycloalkyl, or substituted or unsubstituted 5-membered heteroaryl;

R¹¹ is independently hydrogen, substituted or unsubstituted alkyl, substituted or unsubstituted haloalkyl, substituted or unsubstituted heteroalkyl, substituted or unsubstituted aryl, substituted or unsubstituted heteroaryl, substituted or unsubstituted heterocycloalkyl, or substituted or unsubstituted cycloalkyl;

R¹² and R¹³ are each independently hydrogen, substituted or unsubstituted alkyl, substituted or unsubstituted haloalkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or R¹² and R¹³ are joined together with the nitrogen to which they are attached, to form substituted or unsubstituted 3- to 7-membered heterocycloalkyl, or substituted or unsubstituted 5-membered heteroaryl;

each R⁶, R⁷, R⁸, R⁹, R¹⁰, R¹¹, and R¹² group may optionally be substituted with 1 to 3 groups selected from amino, alkyl, alkenyl, alkynyl, alkoxy, cycloalkyl, cyano, haloalkyl, halogen, hydroxyl, heteroalkyl, heterocycloalkyl, nitro, oxo, aryl, alkylaryl, heteroaryl, and heteroalkylaryl; and

n is independently an integer from 0, 1, 2, 3, 4, 5 or 6.

In another aspect the disclosure provides pharmaceutical compositions including the compound of Formula I and a pharmaceutically acceptable carrier.

In another aspect the disclosure provides methods of treating cancer by administering a pharmacologically effective amount of the pharmaceutical composition, including the compound of Formula I and a pharmaceutically acceptable carrier, to a patient in need thereof.

In another aspect the disclosure provides methods for inhibiting JNK kinase by contacting JNK kinase with a compound of Formula I.

In another aspect the disclosure provides methods of preparing the compound of Formula I.

### DETAILED DESCRIPTION OF THE DISCLOSURE

### Definitions

Abbreviations used herein have their conventional meaning within the chemical and biological arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or cyclic hydrocarbon radical, or combinations thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, N-propyl, isopropyl, N-butyl, sec-butyl, tert-butyl, isobutyl, cyclobutyl, pentyl, cyclopentyl, hexyl, cyclohexyl, (cyclohexyl)methyl, cyclopropylmethyl, homologs and isomers of, for example, N-pentyl, N-hexyl, N-heptyl, N-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—, —CH₂CH═CHCH₂—, —CH₂C═CCH₂—, —CH₂CH₂CH(CH₂CH₂CH₃)CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

As used herein, the terms “alkyl” and “alkylene” are interchangeable depending on the placement of the “alkyl” or “alkylene” group within the molecule.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃ and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′-represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. As used herein, the terms “heteroalkyl” and “heteroalkylene” are interchangeable depending on the placement of the “heteroalkyl” or “heteroalkylene” group within the molecule.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, when the heteroatom is nitrogen, it can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively. As used herein, the terms “cycloalkyl” and “cycloalkylene” are interchangeable depending on the placement of the “cycloalkyl” or “cycloalkylene” group within the molecule. As used herein, the terms “heterocycloalkyl” and “heterocycloalkylene” are interchangeable depending on the placement of the “heterocycloalkyl” or “heterocycloalkylene” group within the molecule.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like. As used herein, the terms “haloalkyl” and “haloalkylene” are interchangeable depending on the placement of the “haloalkyl” or “haloalkylene” group within the molecule.

The term “aryl” means, unless otherwise stated, a polyunsaturated, aromatic, hydrocarbon substituent which can be a single ring or multiple rings, which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. For example, pyridine N-oxide moieties are included within the description of “heteroaryl.” A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 3-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent radicals of aryl and heteroaryl, respectively. As used herein, the terms “aryl” and “arylene” are interchangeable depending on the placement of the “aryl” and “arylene” group within the molecule. As used herein, the terms “heteroaryl” and “heteroarylene” are interchangeable depending on the placement of the “heteroaryl” and “heteroarylene” group within the molecule.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover aryls substituted with one or more halogens.

Where a heteroalkyl, heterocycloalkyl, or heteroaryl includes a specific number of members (e.g., “3 to 7 membered”), the term “member” referrers to a carbon or heteroatom.

The term “oxo” as used herein means an oxygen that is double bonded to a carbon atom.

Each of the above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical. Substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R′R′″, —OC(O)R′, —C(O)R′, —CO₂R—C(O)NR″R′″, —OC(O)NR′R″, —NR′C(O)R″, —NR′—C(O)NR″R′″, —NR′C(O)OR″, —NR′—C(NR″R′″)═NR″″, —S(O)R′, —S(O)₂R′, —S(O)₂NR′R″, —NR′SO₂R″, —CN and —NO₂ in a number ranging from zero to (2 m′+1), where m¹ is the total number of carbon atoms in such radical. R′, R″, R′″ and R″″ each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the disclosure includes more than one R group, for example, each of the R groups is independently selected as are each R¹, R″, R′″ and R″″ groups when more than one of these groups is present. When R¹ and R″ are attached to the same nitrogen atom, they can be combined with the nitrogen atom to form a 4-, 5-, 6-, or 7-membered ring. For example, —NR¹R″ is meant to include, but not be limited to, pyrrolidinyl and morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR′C(O)R″, —NR′—C(O)NR″R′″, —NR′C(O)OR″, —NRC(NR′R″R′″)═NR″″, —NRC(NR′R″)═NR′″, —S(O)R′, —S(O)₂R′, —S(O)₂NR′R″, —NR′SO₂R″, —CN and —NO₂, in a number ranging from zero to the total number of open valences on aromatic ring system; and where R′, R″, R′″ and R″″ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CR′R″—, —O—, —NR′—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CR′R″)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R′, R″, and R′″ are independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

An “aminoalkyl” as used herein refers to an amino group covalently bound to an alkylene linker. The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “substituent group,” as used herein, means a group selected from at least the following moieties: (A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The neutral forms of the compounds are regenerated by contacting the salt with a base or acid and isolating the parent compound in the conventional manner. The parent form of the compound differs from the various salt forms in certain physical properties, such as solubility in polar solvents.

Certain compounds of the disclosure can exist in unsolvated forms as well as solvated forms, including hydrated forms. In general, the solvated forms are equivalent to unsolvated forms and are encompassed within the scope of the disclosure. Certain compounds of the disclosure may exist in multiple crystalline or amorphous forms. In general, all physical forms are equivalent for the uses contemplated by the disclosure.

Certain compounds of the disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the disclosure. The compounds of the disclosure do not include those which are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the disclosed compounds are within the scope of the disclosure.

Unless otherwise stated, structures depicted herein are also meant to include compounds which differ in the presence of one or more isotopically enriched atoms. For example, compounds having the disclosed structure except for the replacement of a hydrogen by a deuterium or tritium, or the replacement of a carbon by ¹³C- or ¹⁴C-enriched carbon are within the scope of the disclosure.

The compounds of the disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are encompassed within the scope of the disclosure.

The term “pharmaceutically acceptable salts” is meant to include salts of active compounds which are prepared with relatively nontoxic acids or bases, depending on the particular substituent moieties found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, mono-hydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science, 66:1-19 (1977)). Certain specific compounds of the disclosure contain both basic and acidic functionalities that allow the compounds to be converted into either base or acid addition salts.

In addition to salt forms, the disclosure provides compounds, which are in a prodrug form. Prodrugs of the compounds described herein are those compounds that readily undergo chemical changes under physiological conditions to provide the compounds of the disclosure. Additionally, prodrugs can be converted to the compounds of the disclosure by chemical or biochemical methods in an ex vivo environment. For example, prodrugs can be slowly converted to the compounds of the disclosure when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” in reference to a particular disease includes prevention of the disease.

The symbol >˜w- denotes the point of attachment of a moiety to the remainder of the molecule.

The term “kinase” refers to any enzyme that catalyzes the addition of phosphate groups to a protein residue; for example, serine and threonine kinases catalyze the addition of phosphate groups to serine and threonine residues.

The term “JNK kinase” refers to JNK, also known as C-Jun N-terminal kinases, which is a kinase that binds and phosphosphorylates c-Jun on Ser63 and Ser73 within its transcriptional activation domain, and is a mitogen-activated protein kinase which is responsive to stress stimuli, such as cytokines ultraviolet irradiation, heat shock, and osmotic shock, and is involved in T cell differentiation and apoptosis.

The term “effective amount” of a compound refers a non-toxic but sufficient amount of the compound that provides a desired effect. This amount may vary from subject to subject, depending on the species, age, and physical condition of the subject, the severity of the disease that is being treated, the particular compound used, its mode of administration, and the like. A suitable effective amount may be determined by one of ordinary skill in the art. For example, the disclosed compounds can be administered at a concentration of about 0.1-50 mg/kg, in certain aspects between 0.1 and 5 mg/kg. In some aspects of the disclosure, an effective amount is at least 0.5 mg/kg, for example, 0.5 mg/kg to about 10 mg/kg, or 0.5 mg/kg to about 5 mg/kg. In certain aspects of the disclosure, the disclosed compounds can be administered at a concentration of about 0.5 mg/kg or 1 mg/kg.

The term “pharmaceutically acceptable” refers to a compound, additive or composition that is not biologically or otherwise undesirable. For example, the additive or composition may be administered to a subject along with a compound of the disclosure without causing any undesirable biological effects or interacting in an undesirable manner with any of the other components of the pharmaceutical composition in which it is contained.

As used herein, the term “patient” refers to organisms to be treated by the methods of the disclosure. Such organisms include, but are not limited to, humans. In the context of the disclosure, the term “subject” generally refers to an individual who will receive or who has received treatment for the treatment of a disease, disorder or pathology.

### Allosteric JNK Inhibitors

The compounds of the disclosure are capable of inhibiting kinases, for example, such kinases as JNK, p38, ERK, SRC, or JAK, and may therefore, be useful for the treatment of various disorders, diseases, and pathologies, such as cancer. Accordingly, the compounds of the disclosure, or their pharmaceutically acceptable salts thereof can be used for preparing pharmaceutical compositions, e.g., by combining these compounds and pharmaceutically acceptable carriers. The pharmaceutical compositions can then be used in pharmacologically effective doses for the treatment of various disorders, diseases, and pathologies, such as cancer.

Thus, in one aspect the disclosure provides a compound of Formula I:

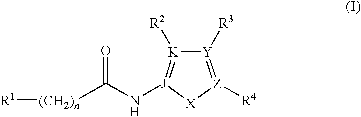

or a pharmaceutically acceptable salt, solvate or hydrate thereof, wherein:

J is independently N or C;

K is independently N or CR²;

Y is independently N or CR³;

Z is independently N or CR⁴;

X is independently O, S, SO, SO₂, or NR⁵;

R¹, R², R³, and R⁴ are each independently hydrogen, halogen, cyano, nitro, hydroxyl, substituted or unsubstituted alkyl, substituted or unsubstituted alkene, substituted or unsubstituted alkyne, substituted or unsubstituted haloalkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, substituted or unsubstituted alkylaryl, substituted or unsubstituted heteroalkylaryl, —(CH₂)ⱼC(═Z)R⁶, —(CH₂)ⱼOR⁶, —(CH₂)ⱼC(O)R⁶, —(CH₂)ⱼC(O)OR⁶, —(CH₂)ⱼNR⁷R⁸, —(CH₂)ⱼC(O)NR⁷R⁸, —(CH₂)ⱼOC(O)NR⁷R⁸, —(CH₂)ⱼNR⁹C(O)R⁶, —(CH₂)ⱼNR⁹C(O)OR⁶, —(CH₂)ⱼNR⁹C(O)NR⁷R⁸, —(CH₂)ⱼS(O)ₖR¹⁰, —(CH₂)ⱼNR⁹S(O)₂R¹⁰, or —(CH₂)ⱼS(O)₂NR⁷R⁸; wherein each j is independently an integer from 0, 1, 2, 3, 4, 5 to 6; and k is independently an integer from 0, 1 to 2; and Z is O, S or NR¹¹;

R⁵ is independently hydrogen or alkyl;

R⁶ is independently hydrogen, substituted or unsubstituted alkyl, substituted or unsubstituted haloalkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁷, R⁸, R⁹ and R¹⁰ are each independently hydrogen, substituted or unsubstituted alkyl, substituted or unsubstituted alkyl-NR¹²R¹³, substituted or unsubstituted alkyl-CONR¹²R¹³, substituted or unsubstituted heteroalkyl, substituted or unsubstituted cycloalkyl, substituted or unsubstituted heterocycloalkyl, substituted or unsubstituted aryl, or substituted or unsubstituted heteroaryl, substituted or unsubstituted aralkyl, substituted or unsubstituted heteroaralkyl, or R⁷ and R⁸, together with the nitrogen to which they are attached, form substituted or unsubstituted 3- to 7-membered heterocycloalkyl, or substituted or unsubstituted 5-membered heteroaryl;

R¹¹ is independently hydrogen, substituted or unsubstituted alkyl, substituted or unsubstituted haloalkyl, substituted or unsubstituted heteroalkyl, substituted or unsubstituted aryl, substituted or unsubstituted heteroaryl, substituted or unsubstituted heterocycloalkyl, or substituted or unsubstituted cycloalkyl;

R¹² and R¹³ are each independently hydrogen, substituted or unsubstituted alkyl, substituted or unsubstituted haloalkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or R¹² and R¹³ are joined together with the nitrogen to which they are attached, to form substituted or unsubstituted 3- to 7-membered heterocycloalkyl, or substituted or unsubstituted 5-membered heteroaryl;

each R⁶, R⁷, R⁸, R⁹, R¹⁰, R¹¹, and R¹² group may optionally be substituted with 1 to 3 groups selected from amino, alkyl, alkenyl, alkynyl, alkoxy, cycloalkyl, cyano, haloalkyl, halogen, hydroxyl, heteroalkyl, heterocycloalkyl, nitro, oxo, aryl, alkylaryl, heteroaryl, and heteroalkylaryl; and n is independently an integer from 0, 1, 2, 3, 4, 5 or 6.

In another aspect the disclosure provides a compound of Formula I, wherein:

J, K, Y, and Z are each independently C;

X is independently S, SO, or SO₂;

R¹ is independently substituted or unsubstituted aryl or substituted or unsubstituted heteroaryl;

R², R³, and R⁴ are each independently hydrogen, substituted or unsubstituted alkyl, or (CH₂)ⱼC(O)NR⁷R⁸;

R⁷ and R⁸ are each independently hydrogen or substituted or unsubstituted alkyl;

j is independently an integer from 0 or 1; and n is independently an integer from 0 or 1.

In another aspect the disclosure provides a compound of Formula I, wherein the compound of Formula I has Formula II:

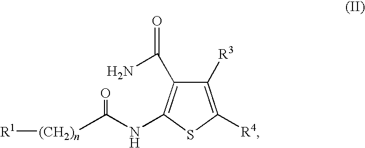

wherein:

R¹ is independently substituted or unsubstituted aryl or substituted or unsubstituted heteroaryl;

R³ and R⁴ are each independently hydrogen, substituted or unsubstituted alkyl; and n is independently an integer from 0 or 1.

In another aspect the disclosure provides a compound of Formula I, wherein R¹ is independently substituted or unsubstituted phenyl, substituted or unsubstituted naphthyl, substituted or unsubstituted thienyl, substituted or unsubstituted benzothienyl, substituted or unsubstituted indolyl, substituted or unsubstituted benzimidazolyl, substituted or unsubstituted thiazolyl, or substituted or unsubstituted isothiazolyl, wherein each R¹ group may optionally be substituted with 1 to 3 groups selected from amino, alkyl, alkenyl, alkynyl, alkoxy, cycloalkyl, cyano, haloalkyl, halogen, hydroxyl, heteroalkyl, heterocycloalkyl, nitro, oxo, aryl, alkylaryl, heteroaryl, and heteroalkylaryl.

In another aspect the disclosure provides a compound of Formula I, wherein the compound of Formula I has formula:

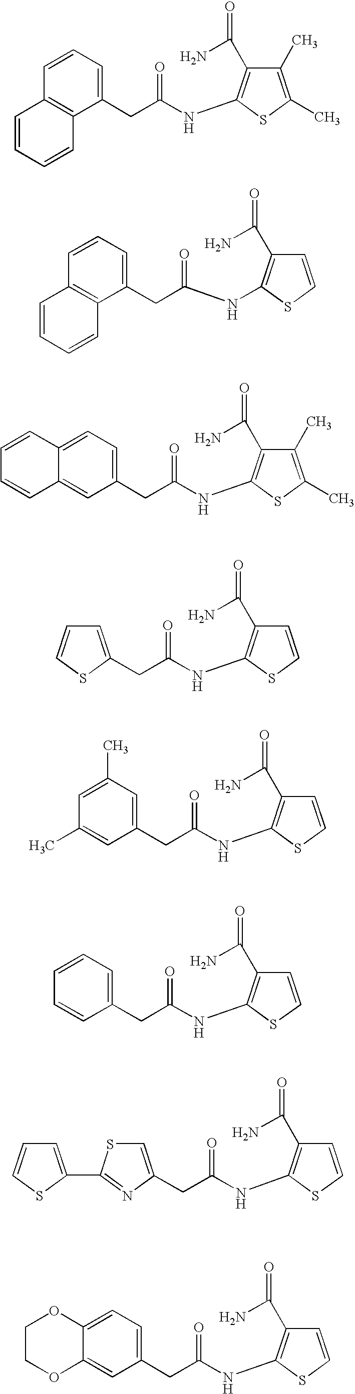

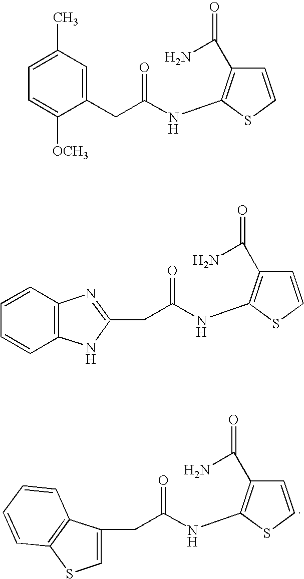

In another aspect the disclosure provides pharmaceutical compositions including the compound of Formula I and a pharmaceutically acceptable carrier.

In another aspect the disclosure provides methods of treating cancer by administering a pharmacologically effective amount of the pharmaceutical composition including the compound of Formula I and a pharmaceutically acceptable carrier to a patient in need thereof.

In another aspect the disclosure provides methods of treating cancer by administering a pharmacologically effective amount of the pharmaceutical composition including the compound of Formula I and a pharmaceutically acceptable carrier to a patient in need thereof, wherein the compound of Formula I is an inhibitor of JNK kinase.

In another aspect the disclosure provides methods for inhibiting JNK kinase by contacting JNK kinase with a compound of Formula I.

In another aspect the disclosure provides methods for inhibiting kinases, the method comprising the step of contacting the kinase with a compound of Formula I.

In another aspect the disclosure provides methods for inhibiting kinases, the method comprising the step of contacting the kinase with a compound of Formula I, wherein the kinase is JNK, p38, ERK, SRC or JAK.

Various synthetic schemes can be designed for preparing compounds of Formula I. To exemplify but not limit the disclosed compounds, in one embodiment the reaction scheme is as shown in Example 1 as provided in the “Examples” section. Other synthetic processes can be designed by those having ordinary skill in the art.

Pharmaceutically acceptable salts of the disclosed compounds may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The disclosed compounds can be formulated as pharmaceutical compositions and administered to a mammalian host, such as a human patient, in a variety of forms adapted to the chosen route of administration, i.e., orally or parenterally, by intravenous, intramuscular, topical or subcutaneous routes. Thus, the disclosed compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2% to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The disclosed compounds may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the methods of preparation may be by vacuum drying and the freeze drying techniques, which yield a powder of the active ingredient plus any additional desired ingredient present in the previously sterile-filtered solutions.

For topical administration, the disclosed compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the disclosed compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the disclosed compounds can be determined by comparing their in vitro activity, and in vivo activity in animal models. Methods for the extrapolation of effective dosages in mice, and other animals, to humans are known to those having ordinary skill in the art who can, for example, be guided by the procedures described in the art, for example as described in U.S. Pat. No. 4,938,949.

Generally, the concentration of the disclosed compounds in a liquid composition, such as a lotion, can be between about 0.1 and 25 mass %, such as between about 0.5 and 10 mass %. The concentration in a semi-solid or solid composition such as a gel or a powder can be between about 0.1 and 25 mass %, such as between about 0.5 and 2.5 mass %.

The amount of the disclosed compounds or an active salt or derivative thereof, required for use in treatment will vary with the particular salt selected but also with the route of administration, the nature of the condition being treated and the age and condition of the patient and will be ultimately at the discretion of the attendant physician or clinician.

The disclosed compounds may also be administered in an amount of between about 0.01 and 25 mg/kg body weight. In certain aspects, the compounds can be administered at a concentration equal to or greater than 1 mg/kg, for example between about 3 and about 20 mg/kg. In other aspects, the disclosed compounds can be is administered at a concentration of between about 5 and about 15 mg/kg. In other aspects, the disclosed compounds can be administered at between about 7 and about 12 mg/kg, for example at 9 mg/kg. It will be understood that the disclosure provides a basis for further studies in humans to more precisely determine effective amounts in humans. Doses used for rodent studies provide a basis for the ranges of doses indicated herein for humans and other mammals.

The route of delivery of the compounds employed by disclosed methods may be determined by the particular disorder. The compounds may be delivered orally, intravenously, intraperitoneally, intramuscularly, subcutaneously, intranasally, and intradermally, as well as, by transdermal delivery (e.g., with a lipid-soluble carrier in a skin patch placed on skin), or even by gastrointestinal delivery (e.g., with a capsule or tablet). Furthermore, the compounds used in the methods of the disclosure, in certain aspects are delivered directly to the brain or certain regions of the brain to activate or inhibit receptors at specific brain sites producing the desirable effect without inhibiting or activating receptors at other brain sites, thus avoiding undesirable side-effects or actions that may counteract the beneficial therapeutic action mediated by the former site(s). The dosage will be sufficient to provide an effective amount of a compound either singly or in combination, as discussed above. Some variation in dosage will necessarily occur depending upon the condition of the patient being treated, and the physician will, in any event, determine the appropriate dose for the individual patient. The dose will depend, among other things, on the body weight, physiology, and chosen administration regimen.

The compounds employed in disclosed methods can be administered alone or in combination with pharmaceutically acceptable carriers, in either single or multiple doses. Suitable pharmaceutical carriers include inert solid diluents or fillers, sterile aqueous solutions, and various nontoxic organic solvents. The pharmaceutical compositions formed by combining one or more compounds with the pharmaceutically acceptable carrier are then readily administered in a variety of dosage forms such as tablets, lozenges, syrups, injectable solutions, and the like. These pharmaceutical carriers can, if desired, contain additional ingredients such as flavorings, binders, excipients, and the like. Thus, for purposes of oral administration, tablets containing various excipients such as sodium citrate, calcium carbonate, and calcium phosphate are employed along with various disintegrants such as starch, and potato or tapioca starch, alginic acid, and certain complex silicates, together with binding agents such as polyvinylpyrolidone, sucrose, gelatin, and acacia. Additionally, lubricating agents, such as magnesium stearate, sodium lauryl sulfate, and talc are often useful for tableting purposes. Solid compositions of a similar type may also be employed as fillers in salt and hard-filled gelatin capsules. Appropriate materials for this purpose include lactose or milk sugar and high molecular weight polyethylene glycols.

The disclosed compounds or one of its pharmaceutically acceptable salts as defined herein, may be useful in the treatment of cancer and manufacturing of a pharmaceutical composition intended for the treatment of cancers, whatever their nature and their degree of anaplasia, in particular including cancers such as melanomas, carcinomas, sarcomas, fibrosarcomas, leukaemias, lymphomas, neuroblastomas, medulloblastomas, glioblsatomas, astrocytomas, angioblastomas, meningiomas, retinoblastomas, prolactinomas, macrobulimia, leiomyosarcomas, mesotheliomas, choriocarcinomas, pheochromocytomas, myelomas, polycythemias, angiosarcomas, extra-skeletal chondrosarcomas, hemangiosarcomas, osteosarcomas, and chondrosarcomas.

By way of example of such cancers, the following can be cited: pancreatic cancer, cancers of the oropharynx, stomach cancer, cancer of the oesophagus, colon and rectal cancer, brain cancer, in particular gliomas, ovarian cancer, liver cancer, kidney cancer, cancer of the larynx, thyroid cancer, lung cancer, bone cancer, multiple myelomas, mesotheliomas and melanomas, skin cancer, breast cancer, prostate cancer, bladder cancer, cancer of the uterus, testicular cancer, non-Hodgkin's lymphoma, leukaemia, Hodgkin's disease, cancer of the tongue, cancer of the duodenum, bronchial cancer, pancreatic cancer and soft tissue cancers, as well as metastatic secondary locations of the aforementioned cancers, such as in the lung, liver and breast.

The disclosed compounds and compositions may be used in combination with one or more chemotherapeutic agents including but not limited to methotrexate, cisplatin/carboplatin; canbusil; dactinomycin; taxol (paclitaxol), antifolate, colchicine, demecolcine, etoposide, taxane/taxol, docetaxel, doxorubicin, anthracycline antibiotic, doxorubicin, daunorubicin, caminomycin, epirubicin, idarubicin, mitoxanthrone, 4-demethoxy-daunomycin, 11-deoxydaunorubicin, 13-deoxydaunorubicin, adriamycin-14-benzoate, adriamycin-14-octanoate, adriamycin-14-naphthaleneacetate, trastuzumab, bevacizumab, OSI-774, or Vitaxin.

The disclosed compounds and compositions may also be useful for treating a neurological disorder or neurodegenerative disease, for example, a disease selected from the group of Alzheimer's disease; fronto-temporal dementia; cerebrovascular disease; stroke; Parkinson's disease; amyotrophic lateral sclerosis; multiple sclerosis; central or peripheral nervous system damage, dysfunction, or complications involving same stemming from edema, injury, or trauma; neurodegenerative changes in postmenopausal women and andropausal men; carpel tunnel syndrome; Charcot-Marie-Tooth disease; diabetic neuropathy; neurofibromatosis; peripheral neuropathy; prion diseases; progressive supranuclear palsy; restless leg syndrome; spinal cord injury; tardive dyskinesia; brain tumors; and neurological developmental disorders including autism, Angelman syndrome and cerebral palsy.

The disclosed compounds and compositions may further be useful for treating disorders wherein the disorder is myocardial infarction, stroke, congestive heart failure, an ischemia or reperfusion injury, arthritis or other arthropathy, retinopathy or vitreoretinal disease, macular degeneration, autoimmune disease, vascular leakage syndrome, inflammatory disease, edema, chronic obstructive pulmonary disorder, shock, transplant rejection, burn, or acute or adult respiratory distress syndrome (ARDS).

When aqueous suspensions of elixirs are desired for oral administration, the compounds may be combined with various sweetening or flavoring agents, colored matter or dyes, and if desired, emulsifying or suspending agents, together with diluents such as water, ethanol, propylene glycol, glycerin, and combinations thereof. For parenteral administration, solutions of preparation in sesame or peanut oil or in aqueous polypropylene glycol are employed, as well as sterile aqueous saline solutions of the corresponding water soluble pharmaceutically acceptable metal salts previously described. Such an aqueous solution should be suitably buffered if necessary and the liquid diluent first rendered isotonic with sufficient saline or glucose. These particular aqueous solutions are especially suitable for intravenous, intramuscular, subcutaneous, and intraperitoneal injection. The sterile aqueous media employed are all readily obtainable by standard techniques well known to those skilled in the art.

Pharmaceutically acceptable salts of the compounds of the disclosure may be obtained using standard procedures well known in the art, for example by causing a reaction between a sufficiently basic compound such as an amine and a suitable acid affording a physiologically acceptable anion. Alkali metal (for example, sodium, potassium or lithium) or alkaline earth metal (for example calcium) salts of carboxylic acids can also be made.

The disclosed compounds can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The disclosed compounds may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In many cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the methods of preparation are vacuum drying and the freeze drying techniques, which yield a powder of the active ingredient plus any additional desired ingredient present in the previously sterile-filtered solutions.

For topical administration, the disclosed compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the disclosed compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used Lo impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the disclosed compounds can be determined by comparing their in vitro activity, and in vivo activity in animal models. Methods for the extrapolation of effective dosages in mice, and other animals, to humans are known to those having ordinary skill in the art who can, for example, be guided by the procedures described in the art, for example as described in U.S. Pat. No. 4,938,949.

Generally, the concentration of the disclosed compounds in a liquid composition, such as a lotion, can be between about 0.1 and 25 mass %, such as between about 0.5 and 10 mass %. The concentration in a semi-solid or solid composition such as a gel or a powder can be between about 0.1 and 25 mass %, such as between about 0.5 and 2.5 mass %.

The amount of the disclosed compounds or an active salt or derivative thereof, required for use in treatment will vary with the particular salt selected, the route of administration, the nature of the condition being treated and the age and condition of the patient, ultimately, with the discretion of the attendant physician or clinician.

### EXAMPLES

For further illustration of various aspects of the disclosure, several specific examples will now be described. It should be understood however that these examples are for illustrative purposes only, and are not intended to limit the scope of the disclosure.

### Example 1

### Synthesis of the Compounds of Formula I

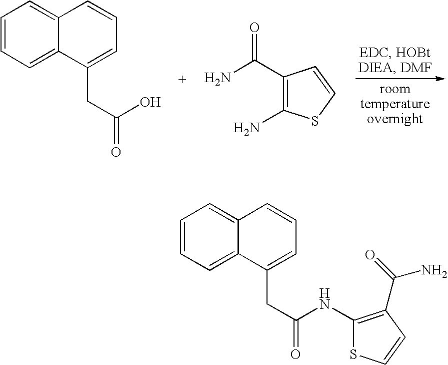

As shown above, the compounds of Formula I may be prepared by standard peptide coupling conditions between a carboxylic acid containing compound and a primary amine containing compound using EDC, HOBT, DIEA in DMF, to afford the amide linked compound of Formula I.

### Example 2

### Properties of Compounds of Formula I

The disclosed compounds of Formula I were tested using Delfia Assay and Kinase Assay and the data for IC₅₀ were obtained. These results are shown in Table I (Kinase assay).

TABLE I
Comparative Results on Inhibition Using Compounds of Formula I

| | | Lantha Screen Kinase |
|---|---|---|
| Compound | MW | Activity Assay IC₅₀ (μM) |
| 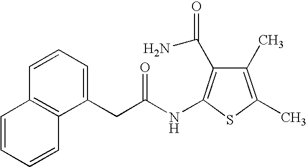 | 338.42 | 18 (1.0 μM in pepJIP1 displacement assay) |
| 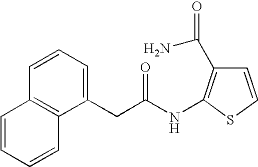 | 310.37 | 5.4 |
| 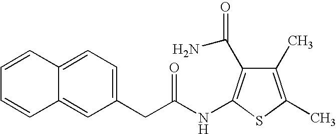 | 338.42 | 5.3 |
| 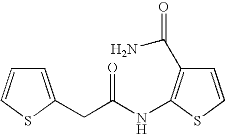 | 266.33 | 3.2 |
| 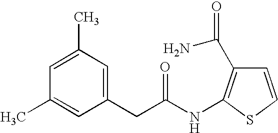 | 288.36 | 1.7 |
| 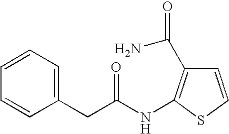 | 260.31 | 1.6 |
| 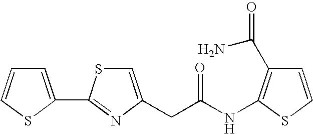 | 349.45 | 60% at 100 μM |
| 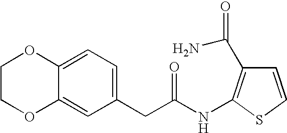 | 318.34 | 2.7 |
| 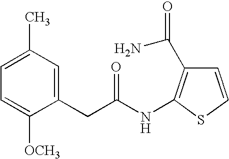 | 304.36 | 2.3 |
| 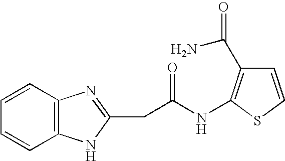 | 300.33 | — |
| 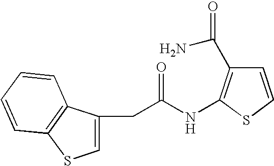 | 316.39 | — |

While the disclosure has been particularly shown and described with reference to several embodiments thereof, it will be understood by those of ordinary skill in the art that various changes in form and details may be made thereto without departing from the principles and spirit of the disclosure, the proper scope of which is defined in the following claims and their equivalents.

What is claimed is:

1. A compound of Formula I: 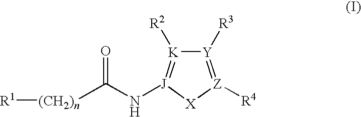
or a pharmaceutically acceptable salt, solvate or hydrate thereof, wherein:
J is independently N or C;
K is independently N or CR²;
Y is independently N or CR³;
Z is independently N or CR⁴;
X is independently O, S, SO, SO₂, or NR⁵;
R¹, R², R³, and R⁴ are each independently hydrogen, halogen, cyano, nitro, hydroxyl, substituted or unsubstituted alkyl, substituted or unsubstituted alkene, substituted or unsubstituted alkyne, substituted or unsubstituted haloalkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, substituted or unsubstituted alkylaryl, substituted or unsubstituted heteroalkylaryl, —(CH₂)ⱼC(═Z)R⁶, —(CH₂)ⱼOR⁶, —(CH₂)ⱼC(O)R⁶, —(CH₂)ⱼC(O)OR⁶, —(CH₂)ⱼNR⁷R⁸, —(CH₂)ⱼC(O)NR⁷R⁸, —(CH₂)ⱼOC(O)NR⁷R⁸, —(CH₂)ⱼNR⁹C(O)R⁶, —(CH₂)ⱼNR⁹C(O)OR⁶, —(CH₂)ⱼNR⁹C(O)NR⁷R⁸, —(CH₂)ⱼS(O)ₖR¹⁰, —(CH₂)ⱼNR⁹S(O)₂R¹⁰, or —(CH₂)ⱼS(O)₂NR⁷R⁸; wherein each j is independently an integer from 0, 1, 2, 3, 4, 5 to 6;
and k is independently an integer from 0, 1 to 2; and Z is O, S or NR¹¹;
R⁵ is independently hydrogen or alkyl;
R⁶ is independently hydrogen, substituted or unsubstituted alkyl, substituted or unsubstituted haloalkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷, R⁸, R⁹ and R¹⁰ are each independently hydrogen, substituted or unsubstituted alkyl, substituted or unsubstituted alkyl-NR¹²R¹³, substituted or unsubstituted alkyl-CONR¹²R¹³, substituted or unsubstituted heteroalkyl, substituted or unsubstituted cycloalkyl, substituted or unsubstituted heterocycloalkyl, substituted or unsubstituted aryl, or substituted or unsubstituted heteroaryl, substituted or unsubstituted aralkyl, substituted or unsubstituted heteroaralkyl, or R⁷ and R⁸, together with the nitrogen to which they are attached, form substituted or unsubstituted 3- to 7-membered heterocycloalkyl, or substituted or unsubstituted 5-membered heteroaryl;
R¹¹ is independently hydrogen, substituted or unsubstituted alkyl, substituted or unsubstituted haloalkyl, substituted or unsubstituted heteroalkyl, substituted or unsubstituted aryl, substituted or unsubstituted heteroaryl, substituted or unsubstituted heterocycloalkyl, or substituted or unsubstituted cycloalkyl;
R¹² and R¹³ are each independently hydrogen, substituted or unsubstituted alkyl, substituted or unsubstituted haloalkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or R¹² and R¹³ are joined together with the nitrogen to which they are attached, to form substituted or unsubstituted 3- to 7-membered heterocycloalkyl, or substituted or unsubstituted 5-membered heteroaryl;
each R⁶, R⁷, R⁸, R⁹, R¹⁰, R¹¹, and R¹² group may optionally be substituted with 1 to 3 groups selected from amino, alkyl, alkenyl, alkynyl, alkoxy, cycloalkyl, cyano, haloalkyl, halogen, hydroxyl, heteroalkyl, heterocycloalkyl, nitro, oxo, aryl, alkylaryl, heteroaryl, and heteroalkylaryl; and
n is independently an integer from 0, 1, 2, 3, 4, 5 or 6.

2. The compound of claim 1, wherein:
J, K, Y, and Z are each independently C;
X is independently S, SO, or SO₂;
R¹ is independently substituted or unsubstituted aryl or substituted or unsubstituted heteroaryl;
R², R³, and R⁴ are each independently hydrogen, substituted or unsubstituted alkyl, or (CH₂)₃C(O)NR⁷R⁸;
R⁷ and R⁸ are each independently hydrogen or substituted or unsubstituted alkyl;
j is independently an integer from 0 or 1; and
n is independently an integer from 0 or 1.

3. The compound of claim 1, wherein the compound of Formula I has Formula II: 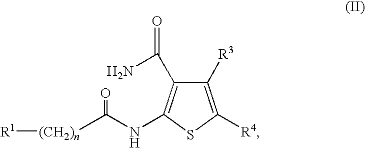
wherein:
R¹ is independently substituted or unsubstituted aryl or substituted or unsubstituted heteroaryl;
R³ and R⁴ are each independently hydrogen, substituted or unsubstituted alkyl; and
n is independently an integer from 0 or 1.

4. The compound of claim 3, wherein R¹ is independently substituted or unsubstituted phenyl, substituted or unsubstituted naphthyl, substituted or unsubstituted thienyl, substituted or unsubstituted benzothienyl, substituted or unsubstituted indolyl, substituted or unsubstituted benzimidazolyl, substituted or unsubstituted thiazolyl, or substituted or unsubstituted isothiazolyl, wherein each R¹ group may optionally be substituted with 1 to 3 groups selected from amino, alkyl, alkenyl, alkynyl, alkoxy, cycloalkyl, cyano, haloalkyl, halogen, hydroxyl, heteroalkyl, heterocycloalkyl, nitro, oxo, aryl, alkylaryl, heteroaryl, and heteroalkylaryl.

5. The compound of claim 1, wherein the compound of Formula I has formula:

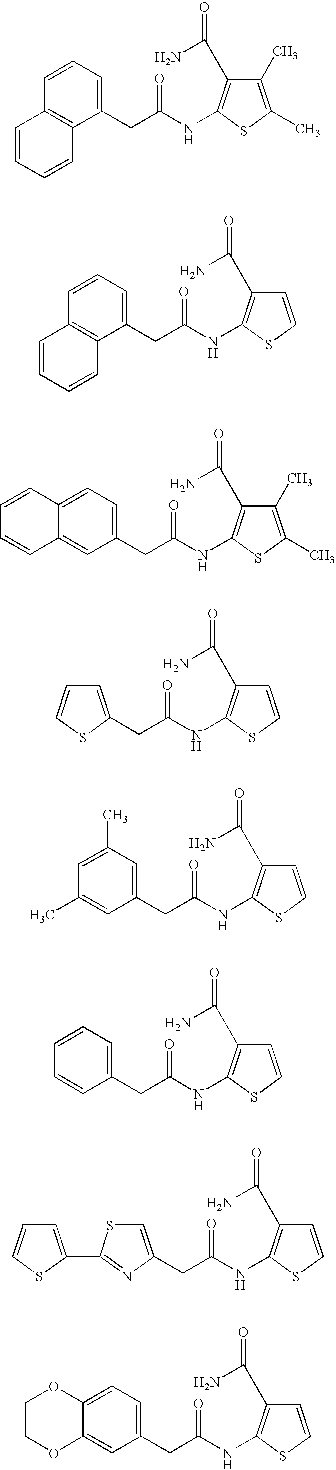

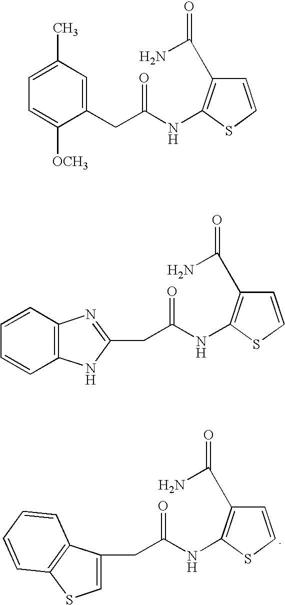

6. A pharmaceutical composition comprising the compound of Formula I of claim 1 and a pharmaceutically acceptable carrier.

7. A method of treating cancer, the method comprising the steps of administering a pharmacologically effective amount of the pharmaceutical composition of claim 6 to a patient in need thereof.

8. The method of claim 7, wherein the compound of Formula I is an inhibitor of JNK kinase.

9. A method for inhibiting JNK kinase, the method comprising the step of contacting JNK kinase with a compound of Formula I of claim 1.

10. A method for inhibiting kinases, the method comprising the step of contacting the kinase with a compound of Formula I of claim 1.

11. The method of claim 10, wherein the kinase is JNK, p38, ERK, SRC or JAK.
